(19) United States
(12) Patent Application Publication
(10) Pub. No.: US 2026/0033902 A1
(43) Pub. Date: Feb. 5, 2026

(54) MEDICAL REMOTE CONTROL, MEDICAL ROBOT WITH INTUITIVE CONTROL, AND CONTROL METHOD

(71) Applicant: B. Braun New Ventures GmbH, Freiburg im Breisgau (DE)

(72) Inventors: Sebastian Zepf, Wurmlingen (DE); Ann-Kathrin Huber, Moenchweiler (DE); Alisa Post, Immenstadt (DE); Shu Chuan Yao, Stuttgart (DE)

(21) Appl. No.: 18/998,192

(22) PCT Filed: Jul. 24, 2023
(86) PCT No.: PCT/EP2023/070455

§ 371 Date: Jan. 24, 2025

(30) Foreign Application Priority Data
Jul. 26, 2022 (DE) 10 2022 118 710.1

(51) Int. Cl. A61B 34/37; A61B 34/00; B25J 13/06; G16H 40/63

(52) U.S. Cl. CPC A61B 34/37; A61B 34/74; B25J 13/065; G16H 40/63; A61B 2034/742; A61B 2034/744

(57) ABSTRACT

A remote control is used to control a medical instrument or device, such as a robot with an end effector. The remote control has a housing, an actuation button, and an input unit that detects force applied to the input unit and outputs a control signal. The actuation button can be arranged at a distance of less than 10 cm from the input unit. A storage unit stores a current control state of the remote control. A control unit processes a selection signal and the control signal, determines and changes a control state, determines a control command, and transmits the command via a data interface for controlling a position and/or orientation of an end effector and/or of a robot arm segment and/or a change in a representation. A medical robot, assistance system, control method and computer-readable storage medium can be used with the remote control.

### CROSS-REFERENCE TO RELATED APPLICATIONS

This application is the United States national phase entry of International Application No. PCT/EP2023/070455, filed on Jul. 24, 2023, and claims priority to German Application No. 10 2022 118 710.1, filed on Jul. 26, 2022. The contents of International Application No. PCT/EP2023/070455 and German Application No. 10 2022 118 710.1 are incorporated by reference herein in their entireties.

### FIELD

The present invention relates to a medical remote control for controlling a medical instrument, in particular a surgical instrument, or a medical, digitally controllable device, in particular a medical collaborative robot with a medical end effector. The present invention also relates to a medical robot, a surgical assistance system, a control method and a computer-readable storage medium and computer program.

### BACKGROUND

Nowadays digital and/or digitally controllable medical instruments or devices frequently require, during a surgical procedure, a remote control by a surgeon or other qualified medical personnel from a sterile field close to the surgical site. The surgeon, however, usually needs both hands at the same time to operate the surgical instruments currently in use.

An acoustic remote control of the instrument or device by means of voice and voice recognition which outputs a corresponding control command and/or control signal currently still suffers from a lack of robustness, as the voice command is not recognized reliably and one-to-one and converted to a control command due to different pronunciations, an operation mask or any other background noise, for example. Therefore, voice control mostly fails due to the need to ensure the patient's safety. In addition, if the surgeon's concentration is too low or an unintentional statement is made, a control command can be triggered inadvertently.

If, on the other hand, keys or a touch display of a separate station, such as on a tower, are used for a remote control, such manual inputs virtually always require that the surgeon interrupts the operation to perform the appropriate remote control.

Furthermore, in the field of medicine or medical engineering, automation with accompanying integration of digitally controllable technical devices continues to gain importance. During a surgical procedure, robots which assist in particular a precise minimally invasive procedure are increasingly used. Accordingly, the robot is not only provided as a standalone robot which is the only one to carry out the operation, but the robot is increasingly used as a collaborative robot (cobot), i.e., as an assisting or supporting robot, directly in the surgical field, which interacts with qualified medical personnel, in particular with the surgeon. In this way, the collaborative robot can carry out parts of surgical procedures and hands over another surgical procedure to a surgeon as required.

For such interaction between humans and robots it is important to enable the user to appropriately control and/or guide an end effector or a part, a robot arm segment or an assembly group of the robot using an input means/input device. According to the present prior art, operating keys are provided for this purpose, for example, wherein typically a pair of keys for each axis in space is used, such as one key for a movement along the x-axis in the positive direction and one key for the movement along the x-axis in the negative direction. The keys can be provided in an external control panel or at any other place where the user interacts with the robot as a kind of remote control. In the cases in which the input device is attached to a place other than the movable segments of the robot, haptic feedback of the robot control is completely lost, however.

In addition, when operating keys are used, the surgeon is required to achieve a high level of intellectual connection between a coordinate system of the end effector and a coordinate system of the control device with the actuation buttons, which is exhausting and increases error-proneness during the surgical procedure. Although it is currently possible to display an extracorporeal image of the operating environment and/or the surgical procedure area on a separate monitor and to control the robot on the basis of the extracorporeal image, but this requires constant head movement by the surgeon which is exhausting. Also, such a modality is not suited for a rough positioning of the end effector, and a user can easily lose the spatial overview. Further, there is the intellectual challenge and the effort of orientation to be made while the information, in particular that of the position and the orientation of the end effector relative to the patient, merge mentally. This is a great challenge in particular to personnel involved in the surgery having an insufficiently trained spatial imagination and makes linking of information more difficult.

Also, a joystick that translates an input joystick movement into a movement of the medical robot and controls the latter can be provided on an external control panel.

Said foregoing remote controls and input means have the drawback that they are insufficiently flexible when being integrated in a surgical procedure and at appropriate places in the operating theater and, apart from a lack of intuitive operation, also lack a correspondingly wide range of functional options.

### SUMMARY

Therefore, the targets and objects of the present invention are to avoid or at least to reduce the drawbacks known from prior art and to provide in particular a medical remote control, a medical robot, a medical control system, a surgical assistance system, a medical control method as well as a computer-readable storage medium and a computer program that admits control of a medical instrument or device in a very simple and intuitive manner and has a particularly compact design and can be arranged flexibly in the operating environment. It is another partial object to provide a remote control for preferably universal use by which different medical instruments and devices can be controlled. Another partial object can be perceived in providing a feasible and sterilizable remote control and a medical robot with remote control which hardly affects the usual working environment when used in a sterile room. A further partial object can be perceived in admitting only a limited selected movement of an end effector or a robot arm segment or an assembly group of the robot, where needed, in order to enable precise control during a surgical procedure and to strictly prevent unintentional detrimental movements of the robot.

A central idea of the present invention can be perceived in making available an integration and a combination of an input means, in particular of a joystick with six degrees of freedom as control signals (6-DoF joystick) and an actuation button (a push-button), in particular in the area of a robot (end) effector for operating a medical system. The actuation button (push-button) allows to change a control state (an operating mode) of the input means, in particular of the joystick, for a versatile use of the input means. The actuation button (push-button) thus enables the user to change an operating mode of the input means, in particular of the joystick, so that the remote control and the input means can be used even more flexibly. Typically, a joystick has a spring-like behavior and returns to its original position again without any application of force (zero axis).

In other words, the present medical remote control is essentially based on a combination of an input means, in particular a joystick with six degrees of freedom, and one (single) key or actuation button (viz. in particular exactly one single actuation button). In particular, they are integrated into a robot guide system/robot control system to guide/control an end effector and to operate the input means, in particular the joystick, in different control states/modes so as to allow for the control of additional functions. The actuation button and the joystick are integrated in the same housing and constitute a uniform (insulated) remote control module and/or are integrated in the same base body and are relatively close to each other so that the actuation button can be ergonomically reached from the input means, in particular from the joystick, by stretching only one finger of the hand instead of having to move the whole hand away from the joystick. The distance between the input means and the actuation button is less than 10 cm to ensure that. Both the actuation button (the push-button) and the input means, in particular the joystick, are connected to a central control unit (virtually as software applications) in order to allow the assignment to different functions of the system and to output a control command based on a control state in combination with the control signal of the input means.

The term “control command” defines a control command deposited in the remote control which can be processed and executed by a medical instrument or device. For example, said control command can be “moving to X by the amount A” of a robot end effector which the robot appropriately implements, and the robot moves the robot end effector by said amount A in the X direction.

In other words, in the present case a medical remote control is disclosed for controlling a medical instrument, in particular a surgical instrument or a medical, digitally controllable device, in particular a medical collaborative robot with a medical end effector. The remote control comprises: a housing with a fastening portion which delimits at least an electronics unit of the remote control relative to surroundings, an actuation button (push-button) arranged in or on the housing, in particular exactly one single actuation button serving to create a selection signal by way of manually actuating the actuation button; an input means with a zero axis protruding from the housing to the outside and being customized to detect at least one force applied to the input means transversely to the zero axis in at least two opposite directions as an input, and to output a control signal, wherein the actuation button is arranged at a predefined maximum distance, in particular a distance of less than 10 cm, from the input means in order to allow manual actuation using the fingers on one hand without having to move a hand or a thenar eminence. Further, the remote control comprises a storage unit in which a current control state of the remote control is stored, and a control unit which is customized to process the selection signal and the control signal and to determine and store a control state on the basis of the selection signal of the actuation button, and to determine an associated control command on the basis of the control state and on the basis of the control signal from the input means and transmit this determined control command via a data interface, in particular for controlling a position and/or orientation of an end effector and/or of the robot arm segment and/or a change in a representation.

The term “zero axis” defines an axis of a zero position or idle position of the input means in which no manually applied (input) force acts upon the input means. In particular, the zero axis of a type of axis of symmetry of the input means which allows at least an input in two opposite directions transversely relative to the zero axis. In particular, the zero axis serves for orientation of an input of the input means.

The term “end effector” in the present invention refers to a device, an instrument or similar medical means which during a surgical procedure on a patient can be used for performing the procedure. In particular, the following can be considered to be an end effector: an instrument, a medical device such as an endoscope or a suction tube, an optical device with a visualization axis, a pointer having a distal tip for a surgical navigation, and others. The term “distal tip” describes the distal area of the pointer that tapers starting virtually from a base shape at a distance perpendicularly to the longitudinal axis, in particular in the diameter, and along the continuous tapering forms a distal end face for a palpation from the tissue.

The term “position” refers to a geometric position in the three-dimensional space indicated in particular by means of coordinates of a Cartesian coordinate system. In particular, the position can be indicated by the three coordinates X, Y and Z.

The term “orientation” in turn refers to an alignment (such as with the position) in space. It can also be said that the orientation indicates an alignment with direction and/or rotation information in the three-dimensional space. In particular, the orientation can be indicated by means of three angles.

The term “pose” comprises both a position and an orientation. In particular, the pose can be indicated by means of six coordinates, three position coordinates X, Y and Z as well as three angular coordinates for the orientation.

Consequently, it is a basic idea for the medical remote control to provide a remote control with an actuation button/an (actuation) key, in particular in the form of a single push-button, in combination with an input means, in particular a joystick with six degrees of freedom, and having a communication interface in order to control an external device having a corresponding external communication interface/user interface. In particular, a standardized communication interface with a standardized protocol is used to render the remote control compatible with a plurality of instruments and devices and to use it universally. A specifically adapted control unit processes the actuation of the actuation button and the input (control signal) of the input means and outputs a corresponding control command directly or indirectly to the medical instrument or device. The medical instrument or device which is digitally controllable, in turn, can perform a corresponding control based on the control command. The surgeon can in this way perform a remote control by means of a haptic actuation of the actuation button and a haptic application of force to the input means. The specific remote control with a specifically configured control unit allows in particular to change a control state by the actuation button in order to transmit different control commands on the basis of the same control signal of the input means.

Any disclosure in connection with the medical remote control is also applicable to the medical robot, the control system and the (control) method, just as any disclosure in connection with the medical robot, the control system and the method is also applicable to the remote control. The features of remote control, medical robot, control system and control method can be exchanged.

Advantageous embodiments shall be explained particularly as follows.

Preferably, the input means can have a deflectable lever with a longitudinal lever axis which is deflectable about a deflection point located on the zero axis relative to the zero axis by a deflection angle and which, without any force applied, swivels back to its zero position coaxially to the zero axis, the input means being designed in particular as a joystick to detect an input in at least four directions, preferably six degrees of freedom.

In particular, a 3D mouse which provides three rotational inputs in addition to three axial inputs as an input can be used as an input means. Hence, in one embodiment, a 3D mouse can be used as a joystick for controlling the robot. One example of a 3D mouse is a product marketed by 3Dconnexion under the registered trademark SPACEMOUSER Compact (having a Z axis perpendicularly to the support surface).

According to another preferred embodiment, a force-moment sensor/force-torque sensor can be used as an input means which, with three axial inputs and three rotational inputs, has six input degrees of freedom. Therefore, one option of interacting with the end effector is the use of a force-torque sensor as an input means in a corresponding control state (robot movement control).

In accordance with another aspect of the disclosure, the remote control can be designed to be sterile or sterilizable. In particular, it includes a fluid-tight cover of materials adapted to be sterilizable in order to be sterile or sterilizable. In particular, the remote control itself is in sterile packaging in order to open the packaging not before the place of use and to safely utilize the remote control there. Consequently, in particular, the remote control has a sterile or sterilizable design, either for single use or for recycling. Preferably, the remote control can be a sterile disposable article/disposable product.

In particular, the housing can be cuboid-shaped and on its front side both the input means and the actuation button are provided and on its (remote) rear side the fastening portion is arranged. Further, the housing can preferably have a fluid-tight design to form a barrier against the surroundings. According to another embodiment, the input means can be arranged symmetrically on and protrude from one side of the housing. Preferably, when both the input means and the actuation button are provided on the front side, a display can further be arranged on the front side so that both the input and the output of the remote control are on one side only.

Preferably, the fastening portion of the housing may include a magnet to mount the housing to a metallic portion or a counter-magnet on the robot. The magnet is preferably coated, in particular surrounded or covered, with rubber or plastic material to the outside (i.e., facing the robot when mounted) to prevent a sterile barrier, in particular a sterile cover of the robot, from being damaged by the magnet, for example by a sharp edge or material abrasion. Preferably, the material selected is a sterilizable rubber or plastic material. The housing with the fastening portion containing the magnet is particularly designed to be hermetically fluid-tight or sealed toward outer surroundings in order to allow, on the one hand, sterilization and, on the other hand, to avoid gaps for germ formation, the rubber coating over the magnet preferably forming a section of the hermetic design. Preferably, the plastic material selected is a soft plastic material having a low hardness of 1 Mohs to prevent the sterile barrier from being damaged. A magnetic fastening is of advantage as an already existing sterile barrier (such as by a transparent cover film) does not have to be broken. The robot arm, in turn, frequently is metallic in the crucial areas so that flexible coupling is possible.

According to a preferred embodiment, the remote control, in particular the 3D mouse such as a 3D mouse marketed by 3Dconnexion under the registered trademark SPACEMOUSER Compact, may include a coating with a sterile wrapping (drape) which can be fastened/fixed to or around the remote control, preferably using a rubber ring as a fixing means which is specifically designed to be autoclavable. Therefore, in this embodiment, the 3D mouse as a remote-control itself (without a coating) does not have to be sterile and may be a standardized product from the field of consumer goods, for example. Also, the remote control does not have to meet any requirement regarding recycling by such a configuration with a (separate) coating. It is sufficient when the coating with the sterile wrapping (such as a sterile single-use coating or a sterilizable coating) is arranged (e.g., attached, pulled or slipped) over the 3D mouse and is finally fixed, in particular using the rubber ring, before the surgical procedure.

The actuation button as a mode key can also be digital in one embodiment and can be displayed on a touch display, in particular a touch display on an end effector.

The selection signal can be processed directly in the control unit of the remote control. In particular, based on different keystroke patterns as an actuation, an appropriate selection signal of a control state can be determined.

Preferably, the control unit and/or the remote control unit include/s a storage unit in which a current control state of the remote control is stored as a parameter, and the control unit can be adapted to finally determine the control command, on the basis of the current control state, on the one hand, and of the control signal, on the other hand. In particular, a control diagram or a table having a first column of the current control state, a second column of the input signal as control signal and a third column of the associated control command can be deposited in the control unit so that the matching combination of the two parameters generates an unambiguous control command which is output to the medical instrument or device. The control command is determined on the basis of said two parameters. In order to move a robot microscope into any direction, for example, a single click switches between “view processing” and “robot movement” as a control state, and during a robot movement the robot microscope can be moved and oriented in space by a joystick as an input means.

According to one aspect of the present invention, the control unit can be customized to detect a length of time/duration and/or a number of actuations within a defined time interval of the actuation button as an input signal and, based on this, in particular based on an assignment deposited in a storage unit, to change, in particular to set, a control state. Consequently, the control unit detects a kind of stroke pattern when the actuation button is actuated as an input signal and, by way of the stroke pattern, determines an associated control state on the basis of a deposited assignment. The remote control and/or the control unit in this way can differentiate between a short stroke (single click), a long stroke (within a time interval), a double stroke (double click) and a continuous stroke in order to activate different command functions. Thus, a user can switch between functions by differentiating between e.g. a short stroke, a long stroke, a double stroke and a continuous stroke.

According to another alternative aspect of the invention, the storage unit can be provided centrally rather than in the remote control, in particular as a cloud storage (computer cloud/data cloud) and/or as a storage unit of a server and/or as a storage unit of the medical instrument or device, and the control unit can access the data of the central storage unit, in particular transmit and receive data, in particular via the wireless data interface. By providing the storage unit centrally, data can be held available and manipulated centrally. In particular, data consistency is given. Also, an exchange of a medical remote control unit is simplified as the important data are centrally provided and, in the case of exchange, no data transfer from one remote control unit to the next remote control unit is necessary. Also, control of one single instrument or device can be performed by plural remote control units. In addition, maintenance of the data and bug fixing can be performed more efficiently.

According to one embodiment, the remote control may include a display. Preferably, the control unit may be adapted to display the current control state of the remote control on the display. In this way, the surgeon or a user of the remote control always keeps an eye on the current control state. As an option, the active control state (control mode) can also be visualized on the main operation display. Preferably, the control unit can also display the subsequent control state that can be switched to by actuation of the actuation button on the display. In other words, as an alternative or in addition to a display of the remote control, also the instrument or device to be controlled can include a display, in particular, for displaying the current control state. In this way, a surgeon can only look at one display such as a surgery monitor of a surgical microscope, read the control state from there and, corresponding to such control state, perform a haptic movement input of the joystick as input means for control.

In particular, depending on the type of event or a temporary stage of surgery, a software application can automatically switch a control state (mode) of the joystick to change the functionalities/settings as required which are controlled via the joystick.

According to one embodiment, a robot movement state can be selectable as a control state in which the control unit outputs the control signal for controlling a robot, in particular a position and/or orientation of an end effector and/or the robot arm segment, via the data interface. In particular, the term “robot arm segment” in this case refers to a robot part of the robot arm supported between joints. In particular, a longitudinal axis refers to a portion of the longitudinal axis adjacent to the input means, viz. the portion near the input means.

According to a further preferred embodiment, a view manipulation mode can be selectable as a control state which allows the user to set functions of a visualization system, in particular of a zoom and/or a focus.

The control unit can preferably be adapted to switch (toggle) between the robot movement mode and the view manipulation mode in the case of an actuation click as selection signal. In particular, the remote control and/or the multi-purpose joystick thus can have the following two main modes for controlling the surgical guide system between which the user can change: 1.) a robot movement mode that allows the user to move and position the robot and/or the robot system to bring a visualization system such as a surgical microscope to the target area or to position robot-guided instruments as end effectors, for example, and 2.) a view manipulation mode that allows the user to set functions/settings of a visualization system such as zoom and focus.

In particular, when (starting) the remote control, the view manipulation mode is set as standard mode to which it is always returned, in particular after expiry of a period of time without any input to the remote control. For example, when the user presses the actuation button as mode key, the free moving mode of the robot is activated and the robot can be positioned with six degrees of freedom. A second click to the actuation button as mode key brings the user back to the view mode.

According to one embodiment, the remote control can further include a timer/timer function, and the control unit changes, after a preset timer duration, in particular after 10 seconds, to the view manipulation mode or to an inactive mode in which the control unit permits manipulation only after actuation of the actuation key in order to prevent a medical instrument from being inadvertently manipulated. In other words, variants of the remote control and, resp., the system may include a deactivated standard mode in which the input or control signals of the joystick are blocked and/or no control command is output. In contrast to the previous concept of interaction, the first single click onto the mode button then activates the view manipulation mode. After that, the system behaves just as the previous concepts of interaction, with the principal difference that the timer for non-actuation of the joystick brings the system after expiry back to the deactivated mode rather than to the robot movement mode. When the joystick is not actuated for a certain period of time, the system automatically switches back to the view manipulation mode for safety reasons.

In particular, a relation of plural control states can be deposited in the storage unit and the control unit can proceed, on the basis of said relation of control states and a selection signal selected for said relation, to a next different control state corresponding to the relation of the control states. Therefore, in the control unit a relation or a flux diagram of plural control states may be deposited and the control unit may be customized, on the basis of said relation of the control states and a selection signal selected for said relation, to proceed to a next different control state corresponding to the relation of the control states. For example, in the case of the remote control that is connected to a robot arm via the data interface, a control state can be: “move robot arm translationally”. When the actuation button is pressed twice (double click as an input signal), the control unit accesses the deposited relation of the control states and determines, on the basis of the control state and the double click, that the new current control state is to be “rotate robot arm”. In this way, it is possible to change (“toggle”) between control states by only one key and in the respective state to select the appropriate command and transmit it as a control command.

In particular, for each control state either all degrees of freedom of the joystick can be activated as control signals or only determined ones can be activated to control linear functions.

In particular, in one state the control unit can admit exactly three degrees of freedom (3 DoF) of the input means as an input, wherein either
(three) translational axes of the input means, in particular of the 3D mouse, control the (three) translational movements of the robot in the selected frame (translational state), or the (three) rotational axes of the input means control (three) rotational movements of the robot in the selected frame (rotational state).

Consequently, in particular, the 3D can be limited to a mode with three degrees of freedom (3DoF mode) in which either translations or rotations are controlled and the remaining other degrees of freedom are not processed in the control unit.

For fine movements, in particular, the control unit can include additional sub-control states (sub-modes) for the robot movements with restricted degrees of freedom. In particular, the following two sub-control states are deposited:

1.) a focus rotation mode in which only the movement of rotation about a spherical plane having a fixed distance from the focus of the visualization system is activated, while the translational axes are deactivated, and

2.) a vector drive mode in which only the translation movement along defined axes is activated, while all rotational axes are deactivated.

According to another embodiment, the input means may have a puck-shaped or ball-shaped or cube-shaped (manually operable) haptic outer contour as an operating unit. In particular, the input means is a puck-shaped joystick with preferably six degrees of freedom which is attached to the end effector and/or the robot segment. As an alternative, the haptic operating surface may have the shape of a sphere or a cube.

In particular, the input means, in particular the joystick, is permanently connected to the end effector and/or the robot arm and, thus, virtually is an integral part of the robot, in particular of the end effector.

According to a preferred embodiment, the (analogous) input signal from the input means, in particular the joystick, can be used approximately proportional to a deflection of the joystick lever, to modulate the velocity of the movements of the joints.

Preferably, when the actuation button is pressed for more than one second (hold), a control state can change to a sub-control state of the control state and you can change between sub-control states by pressing less than one second. Preferably, the control unit receives signals from the actuation button (at least signals for pressing and releasing) and can identify and differentiate single-click events and double-click and long-press events as well as hold events using a timer.

In particular, whenever the robot movement mode and/or the drive mode of the robot is activated, additional inputs from the actuation button (mode key) can be considered to change between the sub-modes, such as a hold interaction in which the system changes between the sub-modes at a particular frequency, as long as the user presses the key, and stores the currently selected mode when the user releases the mode key. Alternatively, also a double-click may be provided to this end.

The user can preferably return by one single mouse click from each sub-control state/sub-mode directly to the view manipulation mode. When the user then reactivates the robot movement mode by a single click, the previously active sub-mode is activated again.

In particular, a timer (non-actuation timer) can be provided, and the control unit can be adapted to switch the system automatically back to the view manipulation mode after a certain period of time without any actuation of the joystick.

Preferably, the view manipulation mode as a control state can comprise additional sub-control states (sub-modes), in particular the sub-control state of a setting of the light intensity.

Moreover, in the storage unit further control states can be deposited, in particular a state of a control of 2D or 3D navigation views/sections and/or a control of the positioning of an actuatable or motor-operated navigation camera.

In particular, the data interface includes a WLAN interface and/or a Bluetooth interface. Said two wireless data transmission formats are standardized, safe and compatible with most medical instruments and devices.

According to another aspect of the disclosure, the remote control may include an energy storage in the form of a battery, in particular a lithium-ion battery. The remote control and the battery can be particularly adapted to be charged inductively in a contactless manner. In this way, the remote control is self-sustaining.

The objects of the present invention are achieved as regards a medical, in particular surgical collaborative robot to actuate a medical, in particular surgical, end effector, by comprising: a robot base as a local connecting point of the robot; a movable robot arm connected to the robot base and having at least one robot arm segment; the end effector connected to, in particular supported on the robot arm, in particular a terminal side of the robot arm, and having an end effector axis, in particular a visualization device which has a visualization axis, and/or a medical, in particular a surgical instrument, which has an instrument axis; and a robot control unit which is adapted to control the robot arm on the basis of a control command. The medical robot includes, in particular on its end effector and/or on the at least one robot arm segment, the medical remote control according to the present invention and/or according to one aspect of the present invention as an input means that transmits the control command to the robot in order to control at least one position and/or orientation of the end effector and/or of the robot arm segment via the control command.

As an option, the remote control can comprise a cancel key (in particular activatable to a limited extent) which is activated for a short period of time whenever the joystick is actuated in the standard mode of the view manipulation. Said key cancels the most recent changes in order to take account of unintentional touches of the joystick. In particular, the cancel key can be located directly on the robot effector and/or on a separate device/touch display.

The objects of the present invention are achieved, as regards a surgical assistance system for use in a surgical procedure on a patient, by a medical collaborative robot according to the present invention and by a navigation system.

According to one embodiment, the assistance system can be customized to limit a degree of freedom when moving the end effector on the basis of navigation data. For example, the assistance system can admit only a movement along an opening channel in a minimally invasive surgery and can block a rotational movement and any other translational movement.

For better user friendliness, an end effector of a robot can also comprise a display element that can visualize the currently activated mode of the joystick including the use of the different axes. In particular, the display element may be one single LED only for displaying the mode (on/off or else a change of color) or a display for a visualization which also displays a use of the axes.

Further, the objects of the present disclosure are achieved by a control method for a medical robot, in particular for a robot of the present invention, by the steps of: detecting an actuation of the actuation button as a selection signal and changing a control state based on the detected selection signal; detecting an input by an input means having a zero axis as control signal; providing the detected input as control command to a control unit; determining a control command on the basis of the control state and on the basis of the control signal of the input means; and outputting the control command via a data interface, in particular for controlling a position and/or orientation of an end effector and/or the robot arm segment and/or a change in a representation.

Regarding a computer-readable storage medium and regarding a computer program, each of the objects is achieved by the latter comprising commands which, when executed by the computer, cause the computer to carry out the method steps of the control method according to the present invention.

Regarding the medical control system for controlling a medical instrument, in particular a surgical instrument, or a medical, digitally controllable device, the objects are achieved in that it comprises: at least one controllable medical instrument or device that can be digitally driven and includes an external data interface; and at least one medical remote control according to the present invention. The data interface of the remote control is compatible with the data interface of the medical instrument or device. In particular, the control commands associated with the instrument or device are compatible in the remote control. The input signal can be processed directly in the control unit of the remote control. As an alternative, or in addition, the input signal can also be processed in an external control unit such as a server in a surgical control system, and a corresponding control command can be determined.

Preferably, the control system can include at least two, preferably at least three remote controls which control the at least one medical instrument or device. In particular, in the control system therefore plural remote controls can be provided and arranged (positioned) in the surgical field. Said remote controls may permit the same or different control commands, for example.

According to another aspect of the invention, the control system can include a surgical robot with a robot arm as medical device, and the remote control can transmit a translational movement and/or a rotational movement of the robot arm and/or a robot arm configuration as a control command to control the robot arm.

A medical sterile space can include the remote control or the control system of the present invention.

### BRIEF DESCRIPTION OF THE DRAWINGS

In the following, the present invention shall be illustrated in detail by way of preferred embodiments with reference to the accompanying Figures, wherein:

FIG. 1 shows a schematic front view of a medical robot according to a preferred embodiment comprising a remote control according to a preferred embodiment;

FIG. 2 shows a perspective view of a medical robot according to a further preferred embodiment comprising a remote control according to another embodiment;

FIG. 3 shows a schematic view to illustrate a change of a control state after expiry of a timer;

FIG. 4 shows another schematic view of a state diagram with possible changes of the control states;

FIG. 5a shows a schematic view of a joystick with a control state-deactivated;

FIG. 5b shows a schematic view of a joystick with a control state-view manipulation mode (zoom/focus);

FIG. 5c shows a schematic view of a joystick with a control state-robot movement mode;

FIG. 5d shows a schematic view of a joystick with a control state-focus rotation; and

FIG. 5e shows a schematic view of a joystick with a control state-robot vector movement.

The Figures are schematic and are only intended to help understand the invention. Like elements are provided with the same reference signs. The features of the different embodiments can be exchanged.

### DETAILED DESCRIPTION

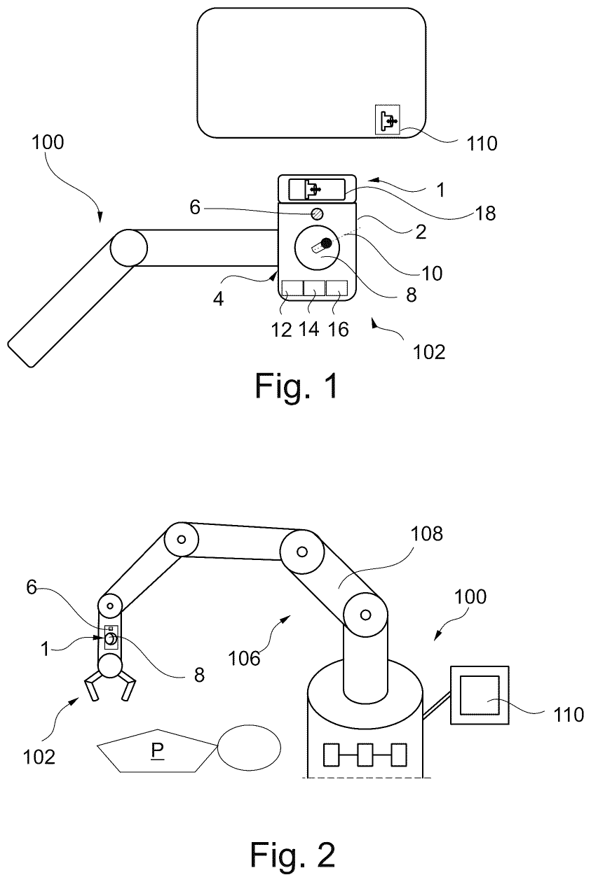

FIG. 1 illustrates in a schematic front view a medical remote control 1 according to a first preferred embodiment which is used in a medical robot 100 according to a preferred embodiment.

The medical remote control 1 for controlling the digitally controllable medical collaborative robot 100 with a medical end effector 102 (behind the remote control 1) has a housing 2 that is fastened, in particular screwed, to the robot 100 via a fastening portion 4. The housing 2 delimits an internal electronics unit of the remote control 1 relative to surroundings. Further, the remote control 1 includes exactly one actuation button 6 arranged on the housing 2 in order to create a (digital electric) selection signal as an actuation signal by way of manual actuation of the actuation button 6, or pressing onto the actuation button 6. Apart from the single actuation button 6, the remote control 1 further includes an input means 8 in the form of a joystick which can be deflected relative to a zero axis 10 to create a control signal depending on the deflection.

Concretely, a force applied to the joystick is detected transversely to and additionally in the direction of the zero axis 10 in six directions (presently an orthogonal coordinate system) as an input, and a corresponding control signal is output in proportional to the deflection.

In the embodiment, the actuation button 6 is arranged at a small distance, i.e., less than 10 cm, from the input means 8 in order to allow a manual actuation using the fingers of one hand without having to move the thenar eminence. Concretely, a longitudinal axis of the actuation button 6 is arranged at a distance of less than 10 cm from the zero axis 10 of the joystick (both axes extend in parallel to each other). Also, the distance can be seen such that a center of the actuation button 6 is at a distance of not more than 10 cm from a center of the input means 8.

In a storage unit 12, relations of control states are deposited and moreover a current control state of the remote control 1 is stored. A control unit 14 is specifically customized to process the selection signal of the actuation button 6 and the control signal of the input means 8 and to determine and store a current control state on the basis of the selection signal of the actuation button 6. Concretely, one can proceed from a first control state of the deposited relation to a second control state according to the relation by a single click.

Further, the control unit 14 is customized to determine an associated control command on the basis of the stored current control state and on the basis of the control signal from the input means 8 and to transmit said determined control command via a data interface 16. In particular, an amplitude of the control command is adapted via the deflection of the joystick (small deflection-small amplitude (move slowly); large deflection-large amplitude (move quickly)). In the present case, the control unit 14 can switch between a view manipulation mode and a robot movement mode as a control state by a single click of the actuation button 6.

In the view manipulation mode, a view is moved corresponding to the joystick on an external display 110 of the robot 100.

In the robot movement mode, on the other hand, the inputs of the joystick as an input means 8 are directly converted to a corresponding movement by transmitting a corresponding control command to the robot 100. When the joystick is moved to the right, the robot is also moved translationally in a specific direction, in particular in exactly the same direction.

Thus, the robot 100 with the remote control is customized to perform control of a position and orientation of an end effector 102 and control of a robot arm segment as well as to perform a change in a representation.

FIG. 2 illustrates another preferred embodiment of a medical robot 100 with a remote control 1 according to the present invention which is used in a surgical assistance system. The robot 100 includes a gripping arm as an end effector 102 and plural robot arm segments 108 of a robot arm 106. A navigation is shown on a display 110. Robot kinematics which is capable of registering also 3D images relative to a patient P, in particular by an external camera, is used as a tracking system.

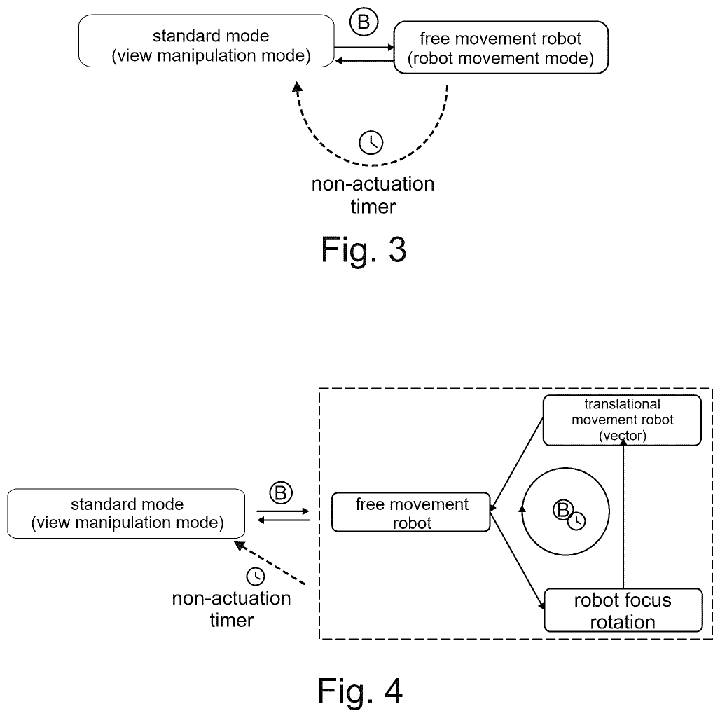

FIG. 3 illustrates an exemplary change of a control state according to a preferred embodiment of a remote control which includes the relations of the control states according to FIG. 3. In a first control state, a standard mode, a view manipulation mode is controlled. Using a single click of the actuation button 6 (shown schematically as B here) one can switch between the standard view manipulation mode and the second control state, i.e. the robot movement mode. However, the control unit also includes a timer having a timer function, in particular of 10 seconds, and automatically returns from the second control state, the robot movement mode, to the view manipulation mode after expiry of the timer. This ensures safety.

FIG. 4 illustrates an exemplary relation of control states according to a further preferred embodiment of a remote control. In contrast to the relation from FIG. 3, the robot movement mode is further divided into sub-control states. Using a long click of the actuation button 8 (more than one second), one can switch anti-clockwise between the three sub-control states: free robot movement (translational and rotational); robot focus function (in order to change a focus of a visualization device such as of a surgery microscope); robot vector movement (in order to admit only a translational movement).

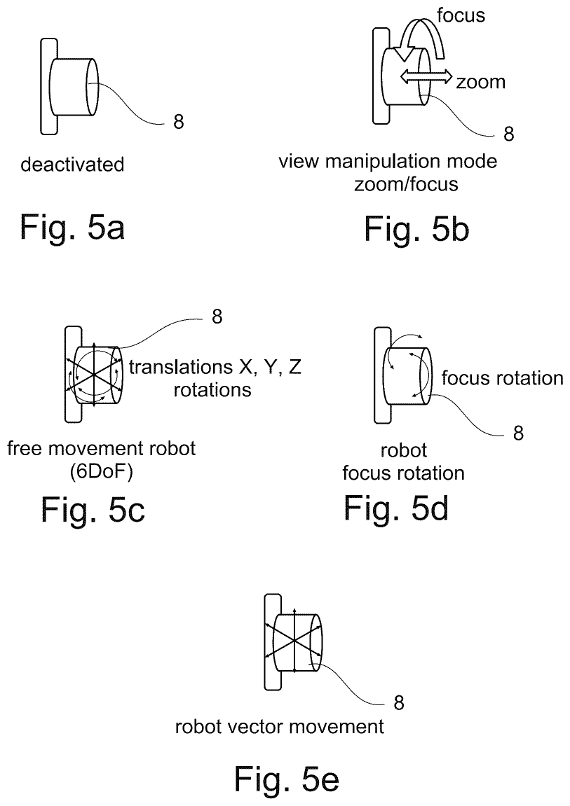

FIGS. 5a to 5e illustrate a joystick as an input means 8 which outputs a corresponding control command depending on the respective current control state and performs a corresponding control.

FIG. 5a schematically illustrates the “deactivated” control state. No control command is output during movement of the joystick.

FIG. 5b schematically illustrates a view manipulation mode to which a zoom is changed by means of pushing or pulling the joystick along its zero axis (control command zoom in-zoom out) and, when rotating the joystick about its longitudinal axis, a focus of a visualization device, such as a surgery microscope, is varied.

FIG. 5c illustrates a (free) robot movement mode as a control state in which the robot or the end effector of the robot is controllable translationally or rotationally corresponding to the input from the joystick, in particular from the 3D mouse.

FIG. 5d schematically illustrates a control state of a focus rotation in which the joystick rotates a focus corresponding to the joystick movement.

Finally, FIG. 5e schematically illustrates a robot vector movement mode as a control state in which only a translational movement of the robot end effector or robot arm segment is performed, wherein a vector by way of which the coordinate system of the joystick aligns is predetermined.

### LIST OF REFERENCE SIONS

1 medical remote control
2 housing
4 fastening portion
6 actuation button
8 input means
10 zero axis
12 storage unit
14 control unit
16 data interface
18 display
20 timer
100 robot
102 end effector
104 robot base
106 robot arm
108 robot arm segment
110 external display
P patient

1-16. (canceled)

17. A medical remote control for controlling a medical device, the medical remote control comprising:
a housing with a fastening portion which delimits at least an electronics unit of the medical remote control relative to surroundings;
an actuation button arranged in or on the housing and serving to create a selection signal by way of manual actuation of the actuation button;
at least one input unit with a zero axis, the at least one input unit protruding outwardly from the housing and being customized to detect at least one force applied to the at least one input unit transversely to the zero axis in at least two opposite directions as an input and to output a control signal, the actuation button being arranged at a distance of less than 10 cm from the at least one input unit in order to allow a manual actuation using a user's fingers on the user's hand without having to move the thenar eminence of the user's hand;
a storage unit configured to store a current control state of the medical remote control; and
a control unit configured to:
process the selection signal and the control signal;
determine and store the current control state based on the selection signal from the actuation button;
determine an associated control command based on the current control state and based on the control signal from the at least one input unit; and
transmit the associated control command via a data interface, the control unit further configured to toggle between a movement mode for controlling a
position and/or orientation of the medical device and a view manipulation mode for controlling one or more view settings of a visualization system.

18. The medical remote control according to claim 17, wherein the medical device is a medical collaborative robot with a medical end effector.

19. The medical remote control according to claim 18, wherein the control unit is configured to transmit the associated control command via the data interface for controlling a position and/or orientation of the medical end effector and/or of a robot arm segment and/or a change in a representation.

20. The medical remote control according to claim 17, wherein the at least one input unit comprises a lever having a longitudinal lever axis, the lever being deflectable about a deflection point located on the zero axis relative to the zero axis with a deflection angle, the lever further being configured to swivel back to a zero position coaxial to the zero axis when no force is applied.

21. The medical remote control according to claim 20, wherein the at least one input unit is a joystick configured to detect an input as a control signal in at least four directions.

22. The medical remote control according to claim 17, wherein the at least one input unit comprises a 3D mouse that provides three rotational inputs as an input and a control signal in addition to three axial inputs.

23. The medical remote control according to claim 17, wherein the at least one input unit comprises a force-moment sensor that provides three axial inputs and three rotational inputs.

24. The medical remote control according to claim 17, further comprising a display, wherein the control unit is configured to show the current control state on the display.

25. The medical remote control according to claim 17, wherein:
the medical device comprises a robot,
the movement mode comprises a robot movement mode,
the robot movement mode is deposited and selectable as a control state in the storage unit, and
the control unit is configured to output a control command in the robot movement mode for controlling the robot.

26. The medical remote control according to claim 25, wherein the control command controls a position and/or orientation of an end effector and/or of a robot arm segment of the robot, via the data interface.

27. The medical remote control according to claim 26, wherein:
the view manipulation mode is deposited and selectable as the control state in the storage unit, and
the one or more view settings of the visualization system comprise a zoom setting and/or a focus setting.

28. The medical remote control according to claim 17, wherein the control unit is configured to switch between the movement mode and the view manipulation mode in response to an actuation click as the selection signal.

29. The medical remote control according to claim 28, further comprising a timer, wherein the control unit is configured, after a predetermined timer period, to change to the view manipulation mode or to an inactive mode in which the control unit only allows manipulation again after the actuation button has been actuated and outputs a control command, in order to prevent the medical device from being inadvertently manipulated.

30. The medical remote control according to claim 29, wherein a relation of plural control states is deposited in the control unit and, based on said relation of plural control states and an input signal selected for said relation of plural control states, the control unit proceeds to a next different control state corresponding to the relation of plural control states.

31. The medical remote control according to claim 30, wherein, when the actuation button is pressed for more than a predetermined period of time, the current control state changes to a sub-control state of the current control state with sub-relations, and by pressing for less than the predetermined period of time, the current control state of the medical remote control is switchable between sub-control states.

32. A medical collaborative robot for actuating a medical end effector, the medical collaborative robot comprising:
the medical remote control according to claim 17;
a robot base as a local connection point of the medical collaborative robot;
a robot arm connected to the robot base and having at least one robot arm segment;
the medical end effector connected to the robot arm and having an end effector axis; and
a robot control unit configured to control the robot arm based on a control command, the medical remote control configured to transmit the control command to the medical collaborative robot in order to control at least one position and/or orientation of the medical end effector and/or of the at least one robot arm segment via the control command.

33. A surgical assistance system to be used for a surgical procedure on a patient, the surgical assistance system comprising:
the medical collaborative robot according to claim 32; and
a navigation system.

34. A control method for a medical robot comprising the steps of:
detecting an actuation of an actuation button as a selection signal and changing a control state based on the selection signal;
detecting an input by at least one input unit with a zero axis as a control signal;
providing the input as a control command to a control unit;
determining a control command based on the control state and on the control signal of the at least one input unit; and
outputting the control command via a data interface.

35. A computer-readable storage medium including commands which, when executed by a computer, cause the computer to carry out the control method according to claim 34.
